(19) United States
(12) Patent Application Publication
(10) Pub. No.: US 2017/0109922 A1
(43) Pub. Date: Apr. 20, 2017

(54) System and method for facilitating assessment of the bowel course and facilitation of transition point detection on cross-sectional radiologic digital images by elimination of air-fluid levels during image post-processing.

(71) Applicant: Michal Kulon, East Setauket, NY (US)

(72) Inventor: Michal Kulon, East Setauket, NY (US)

(21) Appl. No.: 14/884,724

(22) Filed: Oct. 15, 2015

(51) Int. Cl. G06T 15/55; A61B 5/055; A61B 6/00; G06T 19/00; A61B 6/03

(52) U.S. Cl. CPC G06T 15/55; G06T 19/003; A61B 6/032; A61B 6/50; A61B 5/055; A61B 6/481

(57) ABSTRACT

Novel application of pre-existing flood-fill and thresholding algorithms to radiologic images for the purposes of facilitating and accelerating the task of visual radiologic evaluation of the gastrointestinal tract is presented. This invention facilitates evaluation, in a manner that is useful in the contexts of finding a bowel transition point in cases of suspected bowel obstruction, and for facilitating determination of prior unknown surgical bowel alteration (such as Roux-en-Y, Billroth procedures). The invention processes the radiologic 3D or 4D image set, and aims to eliminate or suppress air-fluid or air-contrast levels within the bowel, thereby presenting the radiologist with bowel lumen that is nearly uniform in shade throughout its course, facilitating tracking along the length of the bowel.

### FIELD OF THE INVENTION

Image Processing and Radiology.

### BACKGROUND OF THE INVENTION

In cases of suspected small bowel obstruction, it is desirable for the radiologist or another physician to quickly and easily find the abrupt transition point corresponding to the point of obstruction, between the dilated upstream bowel and the decompressed downstream bowel. Pre-existing methods of visual inspection are relatively time-consuming and can present varying degrees of difficulty, in part due to the presence of multiple air-fluid, air-stool, or air-PO-contrast levels within the lumen of convoluted bowel. Similar difficulties also slow down the visual evaluation of bowel in the context of determining post-surgical bowel course (such as determining whether Roux-en-Y, Billroth, or another procedure has been preformed), and in the context of suspected internal hernia.

While attempting to trace the bowel length for the above purposes, the radiologist must presently visually follow the bowel as its internal contents change multiple times from black (corresponding to air on CT, which floats up into some bowel loops), to gray-white (fluid or oral contrast in loops of the bowel that are relatively lower). Although visualization of air-fluid levels is desirable during initial evaluation, presence of these black-white transitions and resulting inherent perceptual edges within the bowel lumen slow down the process of tracking along the bowel length and make the process of finding a transition point unnecessarily difficult.

Pre-existing flood-fill and thresholding algorithms capable of converting large regions of digital images to another color have been widely used in the fields of graphic processing, but not in combination with radiology PACS system for the purpose of gastrointestinal tract evaluation.

### SUMMARY OF THE INVENTION

The invention changes the heterogeneous appearance of the gastrointestinal tract lumen to a nearly-uniform appearance having similar or same color or gray-level throughout the bowel course, such as by means of converting appearance of air to resemble that of orally-administered contrast material or intrinsic bowel fluid, or by means of performing the the reverse conversion (such as changing voxels corresponding to oral contrast to resemble air). The invention consists of novel application of the pre-existing and widely used flood-fill and thresholding algorithms specifically applied to PACS and radiologic images for the purpose of gastrointestinal tract evaluation, wherein the flood-fill algorithm or thresholding algorithm is combined with a PACS display system (picture archiving and communication system), radiologic work station, or an intermediate processing computer, and provides the means to perform the following functions to the radiologist or another operator. The operator can specify a range of voxel intensities to be replaced with another color or shade such as by selecting a group of one or more voxels from the image or a palette, or means of utilizing an implicit pre-determined range of voxel intensities (such as a range of −600 to −2000 Hounsfields units which are assumed to represent air), thereafter referred to as “canvas substance.” The operator can select a desired new color or shade (thereafter referred to as “paint substance”), such as by clicking on another area of the image (example: fluid within bowel lumen), or selecting from a palette. Flood-fill or thresholding algorithm then processes the set of 3D or 2D radiologic images to cause all or subset of other voxels which have a similar intensity to the “canvas substance” to be changed to another, selectable color or grayscale-level of “paint substance”. This process makes the bowel easier to visually follow by causing the bowel contents to be nearly uniform in appearance.

### DETAILED DESCRIPTION

Those skilled in the art of computer programming and radiologic image processing are able to implement the invention based on the description within the “Summary of Invention” and example herein, using pre-existing descriptions of flood-fill algorithms.

### EXAMPLE EMBODIMENT

The following example is not meant to define the invention's scope, but merely serves to illustrate a sample implementation and demonstrate its usage:
PACS system makes available new software tools, such as entitled “Convert Bowel Air To Fluid” and “Convert Bowel Fluid to Air”, or simply “Flood-Fill.”
When the tool “Convert Bowel Air to Fluid” is activated by the operator, the operator is prompted to select one or more voxels that depict the fluid within the bowel, and optionally one or more voxels that represent air which can also serve as a seed-point for a flood-fill algorithm. The operator is optionally presented a means of changing ranges of voxel intensities that correspond to air.
Software iterates through all or subset of voxels contained in the 3D or 2D image set, and replaces the voxel values that represent air to the value of fluid, and displays the resulting images. This replacement can be optionally restricted to change voxels only in the area of the abdomen, or to process only the voxels that are in vicinity of or directly connected to other similarly-colored voxels (such as by using a 3D implementation of a flood-fill algorithm).

1. System and method comprising a combination of software, computer system, means of displaying radiologic images, means of converting voxels that depict a particular substance (thereafter referred to as “canvas substance”), from one range of voxel intensities to another narrow range of intensities for the purpose of facilitating the radiologic evaluation of the gastrointestinal tract and following along the length of the gastrointestinal tract.

2. Claim 1, wherein the said means of conversion converts the image regions depicting a “canvas substance” to resemble another substance (thereafter referred to as “paint substance”), wherein the “canvas substance” is defined as a member of a set of substances comprising air, gas, fluid, orally-administered contrast, bowel contents, feces, fat, soft tissues, bone, custom substance defined by a custom voxel intensity range, and any mixture thereof.

3. Claim 2, wherein the voxels depicting the “canvas substance” are automatically detected from the image set based on a range of voxel intensities, wherein one bound of the said range of voxel intensities can extend without limit.

4. Claim 3, wherein some ranges of voxel intensities corresponding to possible “canvas substances” are pre-determined.

5. Claim 4, wherein the pre-determined range of intensities corresponding to all gasses is mathematically equivalent to the broad vicinity of the range spanning from −500 Hounsfield Units to a more negative Hounsfield number wherein the said more negative number can extend without limit towards negative infinity.

6. Claim 3, wherein the said system and method are combined with a means for the operator to specify a custom range of voxel intensities, and the said custom range defines the “canvas substance”, in lieu of selecting any particular physical substance.

7. Claim 6, wherein the the said radiologic images are derived from a Computed Tomography Scan.

8. Claim 7, wherein the said “paint substance” is defined as a custom voxel intensity configurable by the operator in lieu of selecting any particular physical substance.

9. Claim 8, wherein the said system and method are combined with a means for the operator to specify the said custom voxel intensity that defines the “paint substance” by means of selecting at least one voxel from the image to use as “paint substance.”

10. Claim 9, wherein the “canvas substance” is similar in appearance on radiologic scan to air, and “paint substance” is similar to a mixture of fluid and orally-administered contrast.

11. Claim 9, wherein the “canvas substance” is similar in appearance on radiologic scan to a mixture of fluid and orally-administered contrast, and “paint substance” is similar to air.

12. Claim 8, wherein the the said radiologic images are derived from an Magnetic Resonance (MRI) scan.
